(19) United States
(12) Patent Application Publication
(10) Pub. No.: US 2025/0043312 A1
(43) Pub. Date: Feb. 6, 2025

(54) SYSTEMS FOR DELIVERING AGENTS INTO CELLS AND METHODS OF USE THEREOF

(71) Applicant: BOARD OF REGENTS, THE UNIVERSITY OF TEXAS SYSTEM, Austin, TX (US)

(72) Inventors: Jeremiah J. GASSENSMITH, Allen, TX (US); Yalini Hansika WIJESUNDARA, Richardson, TX (US)

(21) Appl. No.: 18/701,474

(22) PCT Filed: Oct. 7, 2022
(86) PCT No.: PCT/US2022/077792

§ 371 Date: Apr. 15, 2024

Related U.S. Application Data
(60) Provisional application No. 63/256,075, filed on Oct. 15, 2021.

(51) Int. Cl. C12N 15/88

(52) U.S. Cl. CPC C12N 15/88

(57) ABSTRACT

The present disclosure relates to systems and methods of use thereof for introducing agents into cells. The system and method of introducing an agent into a cell, comprising: providing a composition comprising the agent and a metal organic framework (MOF); and propelling the composition into the cell using compressed gas, wherein the compressed gas comprises CO2.

### CROSS-REFERENCE TO RELATED APPLICATIONS

This application claims the benefit of U.S. Provisional Application No. 63/256,075, filed Oct. 15, 2021, which is expressly incorporated herein by reference in its entirety.

### FIELD

The present disclosure relates to systems for delivering agents into cells and uses thereof.

### BACKGROUND

Biolistic transfection—physically shooting DNA coated metal microparticles into cells—is an established method for introducing foreign genetic material into cells that are otherwise difficult to transfect using traditional methods. Commercial gene guns and the gold or tungsten microparticle “bullets” altogether that carry the DNA into the cells are expensive. Furthermore, there are concerns about their cytotoxicity, biopersistence, and potential for DNA degradation with the genetic material placed on the metal microparticle surface. What is needed are novel tools and methods for delivering biomaterials safely into cells.

### SUMMARY

Previously developed nucleic acid delivery systems have used inert gases or nitrogen rather than CO₂ as particle propellants because of the concerns of CO₂ corrosion. The system provided herein uses CO₂ for creating an acidic environment around the composition comprising a metal organic framework (MOF) (e.g., ZIF-8) when propelling it into the target cell (e.g., through creating an acidic environment in the cellular environment), which quickly dissolves the MOF (e.g., ZIF-8) structure and releases the agent (e.g., a genetic material) contained therein. The release rate of the genetic material from this novel system is much faster with CO₂ than the compressed air. In this case biolistic delivery is in more control when it comes to the release of the genetic material compared to the traditional biolistic delivery with metal microparticles as genetic material detached much faster from the metal surface that have no control in releasing rate.

In some aspects, disclosed herein is a method of introducing an agent into a cell, comprising:
providing a composition comprising the agent and a metal organic framework (MOF); and
propelling the composition into the cell using compressed gas, wherein the compressed gas comprises CO₂.

In some embodiments, the composition is comprised on a film. In some embodiments, the composition is retained within a nozzle. In some embodiments, the film is at the tip of the nozzle.

In some embodiments, the film is a Parafilm®.

In some embodiments, the compressed gas is configured to propel the composition into the cell. In some embodiments, the cell is a plant cell or a mammalian cell.

In some embodiments, a pressure for propelling the composition through the nozzle ranges from about 100 PSI to about 700 PSI. In some embodiments, the pressure for propelling the composition through the nozzle is about 200 PSI or about 500 PSI.

In some embodiments, the compressed gas further comprises nitrogen or an inert gas. In some embodiments, the inert gas is helium.

In some embodiments, the compressed gas further comprises air.

In some embodiments, the agent is selected from the group consisting of a nucleic acid, a protein, a lipid, a small molecule, a bacterium, and a virus.

In some embodiments, the metal organic framework (MOF) is a Cu-based MOF, a Fe-based MOF, a Ni-based MOF, a Co-based MOF, a Zn-based MOF, or a Mg-based MOF. In some embodiments, the MOF is a zeolitic imidazolate framework (ZIF). In some embodiments, the MOF is ZIF-8.

In some embodiments, the agent is encapsulated within the MOF.

In some aspects, disclosed herein is a system for introducing an agent into a cell, comprising:
a composition comprising an agent and a metal organic framework (MOF);
a nozzle configured to retain the composition therein; and
compressed gas comprising CO₂ retained within a chamber that is connected to the nozzle.

In some embodiments, the composition is comprised on a film. In some embodiments, the film is at the tip of the nozzle.

In some embodiments, the compressed gas is configured to propel the composition into the cell. In some embodiments, the cell is a plant cell or a mammalian cell.

In some embodiments, a pressure for propelling the composition through the nozzle ranges from about 100 PSI to about 700 PSI. In some embodiments, the pressure for propelling the composition through the nozzle is about 200 PSI or about 500 PSI.

In some embodiments, the compressed gas further comprises nitrogen or an inert gas. In some embodiments, the inert gas is helium.

In some embodiments, the compressed gas further comprises air.

In some embodiments, the agent is selected from the group consisting of a nucleic acid, a protein, a lipid, a small molecule, a bacterium, and a virus.

In some embodiments, the MOF is a Cu-based MOF, a Fe-based MOF, a Ni-based MOF, a Co-based MOF, a Zn-based MOF, or a Mg-based MOF. In some embodiments, the MOF is a zeolitic imidazolate framework (ZIF). In some embodiments, the MOF is ZIF-8.

In some embodiments, the agent is encapsulated within the MOF.

In some embodiments, the system of any preceding aspect further comprises a pharmaceutically acceptable polymer. In some embodiments, the polymer comprises one or more of polylactic acid (PLA), poly (lactic-co-glycolic acid) (PLGA), tannin, and a starch-based polymer. In some embodiments, the polymer comprises polylactic acid (PLA), poly (lactic-co-glycolic acid) (PLGA), tannin, and a starch-based polymer.

In some aspects, disclosed herein is a method of introducing an agent into a cell comprising introducing the agent into the cell using the system of any preceding aspect.

### DESCRIPTION OF DRAWINGS

The accompanying figures, which are incorporated in and constitute a part of this specification, illustrate aspects described below.

FIG. 1A shows synthesis of DNA loaded ZIF-8 crystals (DsR@ZIF) from 2-methyl imidazole and zinc acetate dihydrate in aqueous conditions at room temperature. FIG. 1B shows biolistic gene transfection process of DsR@ZIF using low-cost MOF-Gun in plants.

FIGS. 2A-2F show SEM micrographs of FIG. 2A) micro ZIF-8, FIG. 2B) micro DsR@ZIF, FIG. 2C) nano ZIF-8, and FIG. 2D) nano DsR@ZIF. E) PXRD of nano and micro formulations of DsR@ZIF compared to synthesized and simulated ZIF-8. FIG. 2F) 1% agarose gel electrophoresis before (left) and after (right) DNase I treatment of DsRed plasmid, DsR@W, and DsR@ZIF-8.

FIG. 3A shows a photograph of MOF-Gun design with components highlighted. Pressurized gas powers the system and is released by triggering the green electronic solenoid. A relay that controls the solenoid is triggered by an electronic firing push button. The DNA is shot from the firing tip onto a sample below. FIG. 3B shows close up of the firing tip containing the loading washers. FIG. 3C shows close up of the loading washers showing the sandwiched parafilm design. FIG. 3D shows schematic of shooting Cy-5 labeled ZIF-8 into 2% agarose gel. FIG. 3E shows cross sectional fluorescence image of the gel showing particle penetration. FIG. 3F shows photograph of gel before shooting Cy-5@ZIF-8. FIG. 3G shows fluorescence image of the gel before shooting Cy-5@ZIF-8. FIG. 3H shows photograph of gel after shooting Cy-5@ZIF-8. FIG. 3I shows fluorescence image of gel after shooting Cy-5@ZIF-8.

FIG. 4A shows schematic of the gene transfection process after shooting DsR@ZIF into plant cells. Confocal micrographs of post gene transfection with FIG. 4B) ZIF-8 (negative control) and FIG. 4C) DsR@W (positive control). Confocal micrographs of FIG. 4D) micro DsR@ZIF and (FIG. 4E) nano DsR@ZIF shooting with compressed air. FIG. 4F and FIG. 4G show that gene expression is seen with micro and nano DsR@ZIF respectively when CO₂ was used as the particle propellant.

FIG. 5A shows Zn²⁺ release profile as measured by ICP-MS after shooting ZIF-8 into DI water with CO₂ gas vs compressed air. The sudden burst of Zn²⁺ reaches its maximum at t=10 mins whereas little free Zn²⁺ is seen in the air sample. FIG. 5B shows pH profiles of solutions when ZIF-8 is fired using compressed air or CO₂ shows a transient mild acidification with a peak at t=10 mins. Following dissolution, anew phase of ZIF (ZIF-C) is formed. SEM images obtained soon after shooting of ZIF-8 with (FIG. 5C) CO₂ and (FIG. 5D) compressed air and after 6 h with (FIG. 5E) CO₂ and (FIG. 5F) compressed air. FIG. 5G shows PXRD patterns of the ZIF-8 crystals after discharging them into water with compressed air and CO₂ propellants after 6 h.

FIG. 6 shows schematic of the MOF-Gun.

FIG. 7 shows agarose gel electrophoresis of DsRed (p), exfoliated micro and nano DsR@ZIF-8 (with EDTA) and the supernatants of micro and nano DsR@ZIF formulations.

FIG. 8 shows standard curve for Zn²⁺ concentration used in ICP-MS.

FIGS. 9A-9F show characterization data obtained from (FIG. 9A) thermogravimetric analysis of DsR@ZIF biocomposites along with pristine ZIF-8 controls (FIG. 9B) N2 adsorption isotherm/BET data of DsR@ZIF biocomposites along with pristine ZIF-8 controls. EDAX of (FIG. 9C) nano ZIF-8 (FIG. 9D) nano DsR@ZIF (FIG. 9E) micro ZIF-8 (FIG. 9F) micro DsR@ZIF for elemental analysis of Carbon-C, Nitrogen-N, Zinc-Zn and Phosphorous-P.

FIG. 10A shows process of gene transfection showing shooting DsR@ZIF into an onion bulb and then incubating in dark conditions for 24 h. FIGS. 10B-10D show confocal images of DsRed fluorescent protein expression with DsR@ZIF with different pressure values.

FIG. 11A shows the Zn²⁺ releasing profile of ZIF-8 resuspend in DI water without shooting. FIG. 11B shows pH profile of the solution of ZIF-8 in DI water. FIG. 11C shows PXRDs of the micro and nano ZIF-8 obtained after 24 h in DI water. SEM micrographs of micro ZIF-8 at FIG. 11D) t=0 and t=24 h. Nano ZIF-8 at FIG. 11F) t=0 and FIG. 11G) t=24 h.

FIG. 12 shows MOF-Gun nozzle.

FIG. 13 shows restriction map and multiple cloning sites of pEGB 35S:DsRed:Tnos (GB0361) provided by Addgene (USA) and amplified in the laboratory.

FIG. 14 shows that OVA-Cy7 was successfully encapsulated in ZIF-8.

FIG. 15 shows schematic of in vivo protein delivery. OVA-Cy7@ZIF was delivered into a flank of mice models using air (0.04% CO₂ and 100% CO₂). The tissue residency of OVA-Cy7@ZIF was monitored through animal imager.

FIG. 16 shows apparatus to measure particle depth penetration following discharge from the MOF jet into skin.

FIGS. 17A-17D show ZIF particles penetration through skin. Cy5@ZIF was biolistically delivered through a mouse skin with compressed air and CO₂. (FIG. 17A) A schematic representation biolistically bombarded ZIF into the mouse using smURFP@ZIF. (Bottom left) The layers of the skin are labeled schematically. (Bottom right) An H&E stained skin cross-section obtained from a male BALB/c mouse to show the actual physiology of the skin. Skin section from FIG. 17B) naïve mice, FIG. 17C) mice shot with ZIF propelled by air, and FIG. 17D) mice shot with CO₂. The fluorescent micrographs show DAPI stained nuclei (top) and the location of the smURFP/Cy5 particles (middle) and their respective overlay images (bottom) with the skin sections labeled as (E) epidermis, (D) dermis, and (S) subcutaneous. The majority of the red fluorescence is in the subcutaneous area.

FIG. 18A shows the tissue residency variation of biolistically bombarded Cy7 labeled OVA in ZIF-8 (OVA-Cy7@ZIF) into stage (iv) human breast cancer model in BALB/c mice with compressed air and CO₂ as the carrier gas. FIG. 18B shows the radiance efficiency changes of the OVA-Cy7@ZIF-8 over time with compressed air and CO₂.

### DETAILED DESCRIPTION

Previously developed nucleic acid delivery systems have used inert gases or nitrogen rather than CO₂ for injecting agent because of the concerns of CO₂ corrosion. The system provided herein uses CO₂ for creating an acidic environment around a composition comprising a metal organic framework (MOF) (e.g., ZIF-8) when propelling the composition into the target cell (e.g., through creating an acidic environment in cytoplasm), which quickly dissolves the MOF (e.g., ZIF-8) structure and releases the agent contained therein. The speed of release from this novel method is much more controllable with the selection of the propellant gas traditional biolistic delivery with metal microparticles that use inert gases or nitrogen for the delivery.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of 20%, +10%, ±5%, or +1% from the measurable value. “Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “antigen” or “immunogen” are used interchangeably to refer to a substance, typically a protein, a nucleic acid, a polysaccharide, a toxin, a pathogen, or a lipid, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “biocompatible” generally refers to a material and any metabolites or degradation products thereof that are generally non-toxic to the recipient and do not cause significant adverse effects to the subject.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges from about 1 nm to about 1000 nm, preferably from about 50 nm to about 500 nm.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.10% and 99% by weight of the total of one or more of the subject compounds based on the weight of the total composition including carrier or diluent.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “copolymer” as used herein refers to a polymer formed from two or more different repeating units (monomer residues). Copolymer compasses all forms copolymers including, but not limited to block polymers, random copolymers, alternating copolymers, or graft copolymers. A “block copolymer” is a polymer formed from multiple sequences or blocks of the same monomer alternating in series with different monomer blocks. Block copolymers are classified according to the number of blocks they contain and how the blocks are arranged. “Therapeutic agent” refers to any composition that has a beneficial biological effect.

Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” refers to the amount of a composition that will elicit the biological or medical response of a tissue, system, animal, or human that is being sought by the researcher, veterinarian, medical doctor or other clinician over a generalized period of time. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of the therapeutic agent as described herein can be determined by one of ordinary skill in the art.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a cancer.

### Systems and Methods

In some aspects, disclosed herein is a system for introducing an agent into a cell, comprising:
a composition comprising an agent and a metal organic framework (MOF);
a nozzle configured to retain the composition therein; and
compressed gas comprising CO₂ retained within a chamber that is connected to the nozzle.

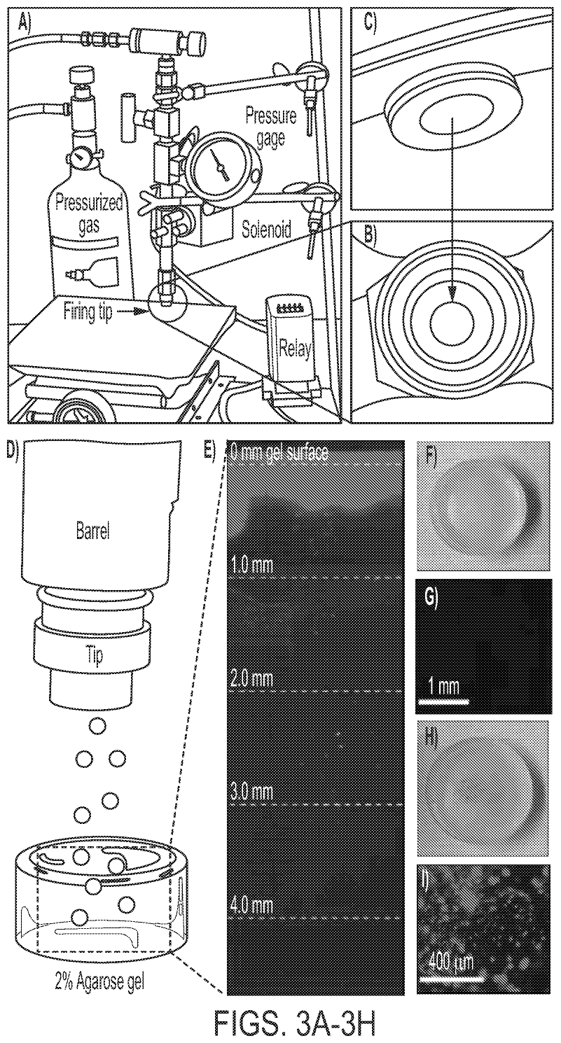

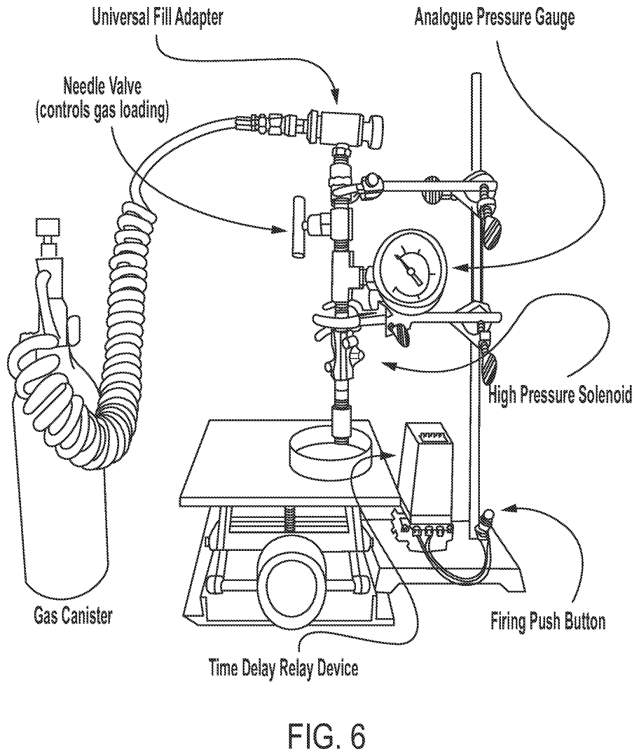

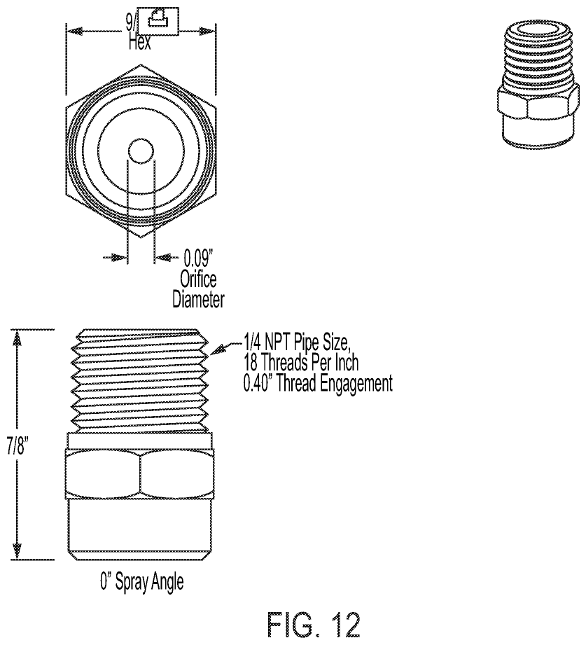

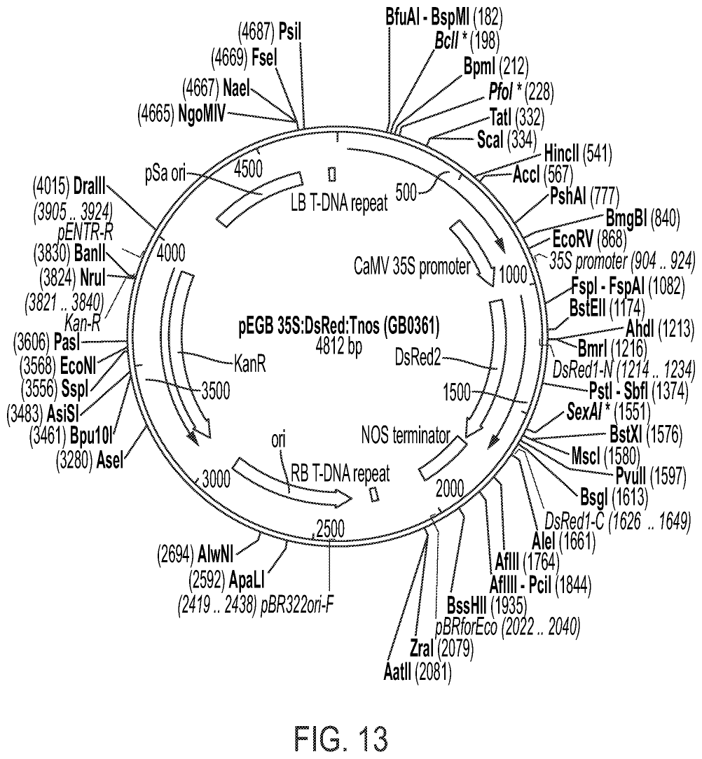

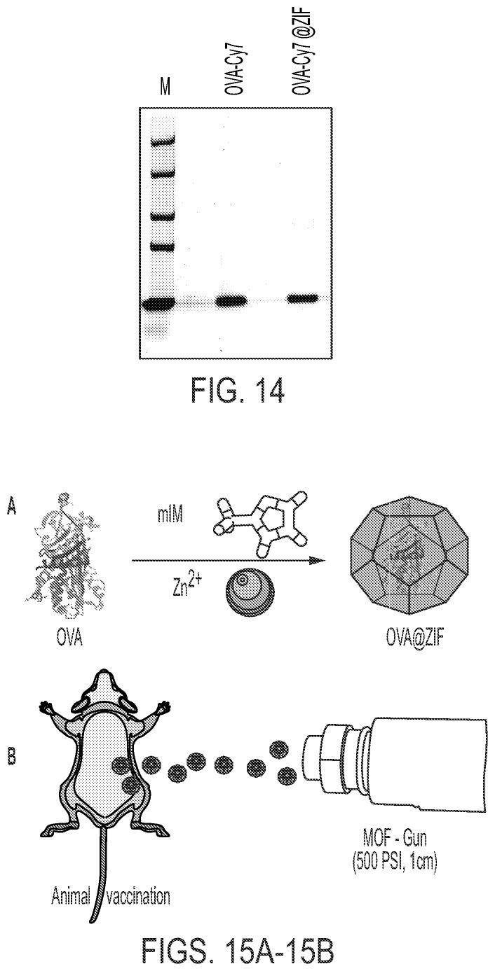

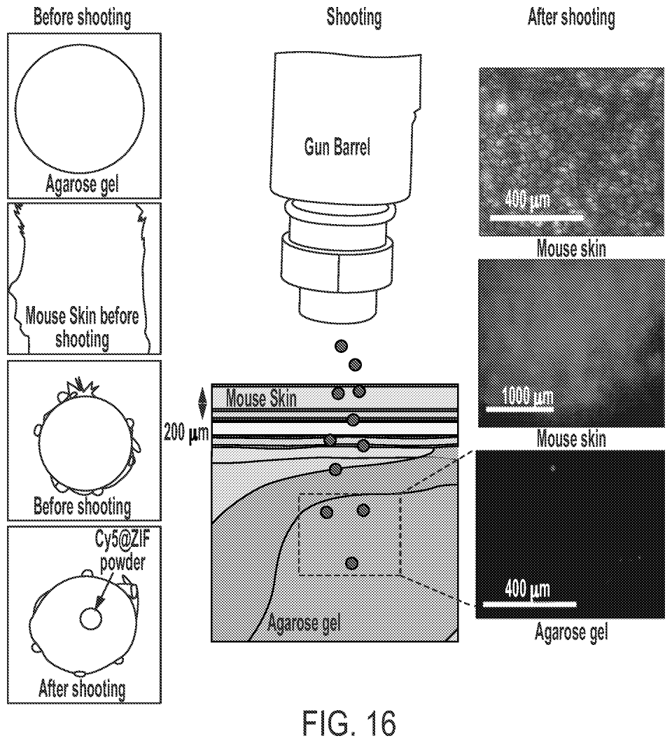

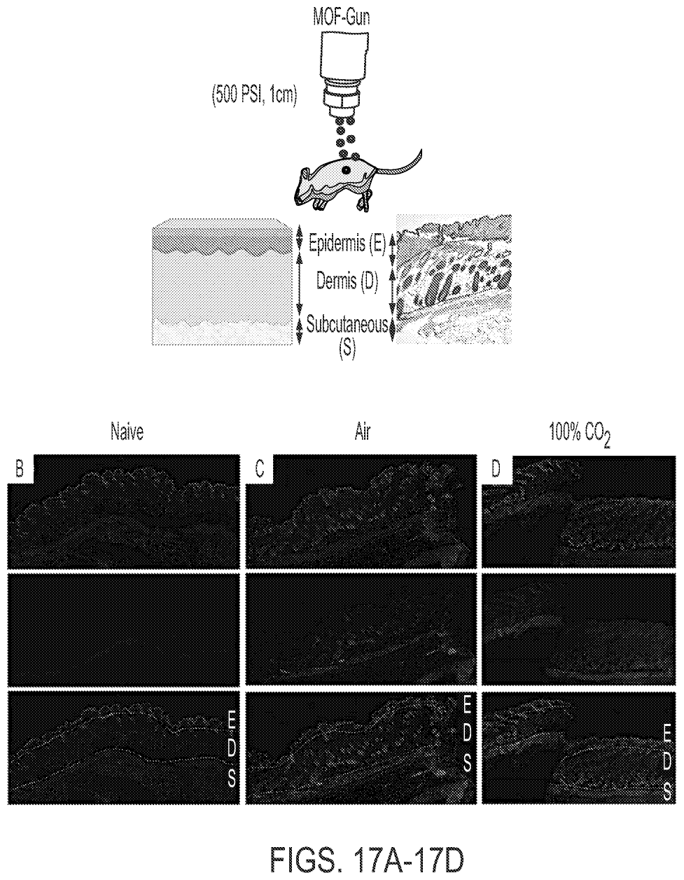

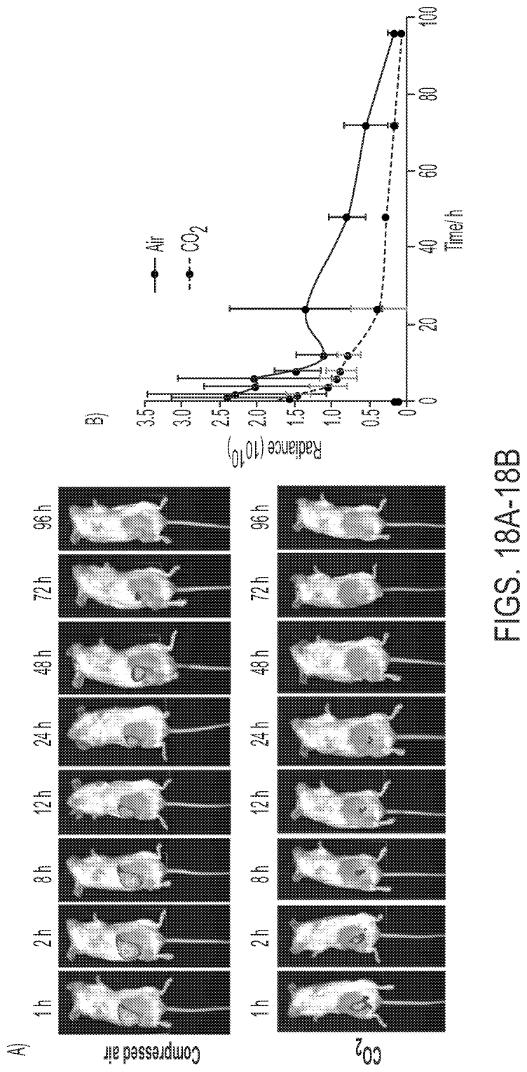

In some embodiments, the composition (for example, a composition in liquid form or solid form) is comprised within a film. In some embodiments, the film is at the tip of the nozzle. In some embodiments, the design of the nozzle with the film is as shown in FIG. 3 and/or FIG. 12. In some embodiments, the system described herein is as shown in FIG. 6. In some embodiments, the film is a Parafilm®.

The pressure of the system provided herein for propelling the composition through the nozzle ranges from about 10 PSI to about 1000 PSI, from about 50 PSI to about 700 PSI, from about 60 PSI to about 500 PSI, from about 80 PSI to about 500 PSI, from about 100 PSI to about 500 PSI, or from about 150 PSI to about 300 PSI. In some embodiments, the pressure of the system provided herein for propelling the composition through the nozzle is about 100 PSI. In some embodiments, the pressure of the system provided herein for propelling the composition through the nozzle is about 200 PSI. In some embodiments, the pressure of the system provided herein for propelling the composition through the nozzle is about 300 PSI. In some embodiments, the pressure of the system provided herein for propelling the composition through the nozzle is about 400 PSI. In some embodiments, the pressure of the system provided herein for propelling the composition through the nozzle is about 500 PSI. In some embodiments, the pressure of the system provided herein for propelling the composition through the nozzle is about 600 PSI. In some embodiments, the pressure of the system provided herein for propelling the composition through the nozzle is about 700 PSI.

In some embodiments, the compressed gas further comprises nitrogen or an inert gas (including, for example, helium (He), neon (Ne), argon (Ar), krypton (Kr), xenon (Xe), or radon (Rn). In some embodiments, the inert gas is helium.

In some embodiments, the compressed gas further comprises air.

As discussed above, the use of compressed air with CO₂ dissolves the MOF structure and releases the agent contained therein at a speed faster (for example, more than about 1.5 times faster, more than about 2 times faster, more than about 3 times faster, more than about 5 times faster, more than about 10 times faster, more than about 20 times faster, more than about 30 times faster, more than about 50 times faster, more than about 100 times faster, more than about 200 times faster, more than about 500 times faster, more than about 1000 times faster, or more than about 5000 times faster) than the systems using inert gases or nitrogen.

The speed of releasing the agent can be adjusted based on the ratio of CO₂ and the inert gas/nitrogen. Accordingly, in some embodiments, the compressed gas described herein comprises CO₂ and an inert gas/nitrogen, wherein the molar ratio of CO₂ and an inert gas/nitrogen is about 1:100, about 1:80, about 1:50, about 1:20, about 1:15, about 1:10, about 1:5, about 1:4, about 1:3, about 1:2, about 1:1.5, about 1:1, about 1.5:1, about 2:1, about 3:1, about 4:1, about 5:1, about 10:1, about 15:1, about 20:1, about 50:1, about 80:1, or about 100:1. In some embodiments, the compressed gas consists of CO₂.

The systems and methods described herein that use compressed air with CO₂ can increase the amount of the agent contained therein (for example, or at least about a 2-fold, or at least about a 3-fold, or at least about a 4-fold, or at least about a 5-fold or at least about a 10-fold increase, or any increase between 2-fold and 10-fold or greater) than the systems using inert gases or nitrogen.

The agent can be encapsulated or embedded within or on the outer surface of the metal-organic framework. “Metal-organic frameworks” (MOFs) are framework materials, typically three-dimensional, self-assembled by the coordination of metal ions with organic linkers exhibiting porosity, typically established by gas adsorption. In some examples, the MOFs discussed and disclosed herein are at times simply identified by their repeat unit as defined below without brackets or the subscript n. A mixed-metal-organic frameworks (MMOF) is a subset of MOFs having two of more types of metal ions.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined, it simply designates repetition of the formula within the brackets as well as the polymeric and/or framework nature of the material. The concept of a repeat unit applies equally to where the connectivity between the repeat units extends into three dimensions, such as in metal organic frameworks, cross-linked polymers, thermosetting polymers, etc. Note that for MOFs the repeat unit may also be shown without the subscript n.

Metal-organic framework can comprise a monovalent, a divalent, a trivalent, or a tetravalent ligand. The MOFs provided herein can grow onto the surface of the agent to encapsulate the agent without affecting the structure and function of the agent. Within these metal-organic frameworks exist pores which may be useful in absorbing another molecule such as a gas. In some embodiments, the metal-organic framework includes metal clusters that comprise a single metal ion, two metal ions, or three or more metal ions. The metal ion can be selected from the group consisting of Group 1 through 16 metals of the IUPAC Periodic Table of the Elements including actinides, and lanthanides, and combinations thereof. Non-limiting examples of suitable metal ions include Li⁺, Na⁺, K⁺, Rb⁺, Be²⁺, Mg²⁺, Ca²⁺, Sr²⁺, Ba²⁺, Sc³⁺, Y³⁺, Ti⁴⁺, Zr⁴⁺, HI⁴⁺, V⁴⁺, V³⁺, V²⁺, Nb³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Ru²⁺, Os³⁺, Os²⁺, Co³⁺, Co²⁺, Rh²⁺, Rh⁺, Ir²⁺, Ir⁺, Ni²⁺, Ni⁺, Pd²⁺, Pd⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Tl³⁺, Si⁴⁺, Si²⁺, Ge⁴⁺, Ge²⁺, Sn⁴⁺, Sn²⁺, Pb⁴⁺, Pb²⁺, AS⁵⁺, AS³⁺, AS⁺, Sb⁵⁺, Sb³⁺, Sb⁺, Bi⁵⁺, Bi³⁺, Bi⁺, and combinations thereof. Some non-limiting examples of metal organic frameworks include those taught by International Publication Number WO2021/097194A1 and U.S. Pat. No. 9,884,309, all of which are incorporated in their entireties herein by reference. The MOF described herein can comprise any metal that can react with CO₂.

In some embodiments, the MOF is a Cu-based MOF, a Fe-based MOF, a Ni-based MOF, a Co-based MOF, a Zn-based MOF, or a Mg-based MOF. In some embodiments, the MOF is a zeolitic imidazolate framework (ZIF). In some embodiments, the MOF is ZIF-8. In some embodiments, the metal-organic framework is a zeolitic imidazolate framework, such as ZIF-8.

Zeolitic imidazolate frameworks (ZIFs), a sub-family of metal-organic frameworks (MOFs), are crystalline materials prepared by self-assembly of metal ions and imidazolate organic linkers. ZIFs adopt zeolitic topologies and display some of the quintessential stability of these classic inorganic materials. Their large pore volumes and surface areas, along with the possibility for chemical functionalization, have led to applications in gas adsorption, separation and catalysis.

In some examples, the MOF described herein comprise pore of which the pore size is large enough for CO₂ to react, but small enough to protect the agent (e.g., proteins or nucleic acids) encapsulated within the MOF from digestive enzymes. “Pores” or “micropores” in the context of metal-organic frameworks are defined as open space within the MOFs. In some embodiments, the MOF described herein has a pore size from about 0.2 to about 1000 nm. In some embodiments, the MOF has a pore size from about 0.2 nm to about 100 nm, from about 1 nm to about 80 nm, or from about 5 nm to about 75 nm. In some embodiments, the MOF have a pore size from about 1 nm, about 2.5 nm, about 5 nm, about 10 nm, about 15 nm, about 20 nm, about 25 nm, about 30 nm, about 35 nm, about 40 nm, about 45 nm, about 50 nm, about 60 nm, about 70 nm, about 80 nm, about 90 nm, or about 100 nm, or any range derivable therein.

The MOF disclosed herein can be self-assembly to form a nanoparticle. In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the nanoparticle has a diameter from about 150 nm to about 175 nm. In some embodiments, the nanoparticle has a diameter from about 135 nm to about 175 nm. The particles can have any shape but are generally spherical in shape.

The amount of an agent that can be present in the nanoparticle can be from about 0.1% to about 90% of its nanoparticle weight. For example, the amount of an present in the nanoparticle can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 50%, about 55%, about 60%, about 65%, about 70%, about 75%, or about 80% of its nanoparticle weight.

In some embodiments, the composition of any preceding aspect further comprises a pharmaceutically acceptable polymer. In some embodiments, the polymer comprises polylactic acid (PLA), poly (lactic-co-glycolic acid) (PLGA), poly(lactic-co-glycolic acid) (PLGA)-block(b)-polyethylene glycol (PEG) and/or polycaprolactone (PCL). Any suitable synthetic or natural biocompatible polymers may be used. Such polymers are recognizable and identifiable by one or ordinary skill in the art. The polymers used herein for are known in the art. See, e.g., U.S, Patent Publication NO: US20170216219A1, incorporated by reference herein in its entirety. In some embodiments, the polymer comprises one or more of polylactic acid (PLA), poly (lactic-co-glycolic acid) (PLGA), tannin, and a starch-based polymer (e.g., starch, amylose, or amylopectin). In some embodiments, the composition is coated with one or more polymers. In some examples, the composition with one or more polymers (e.g, a composition/system coated with one or more polymers) slows the release of the agent contained in the composition/system (e.g., at a speed about more than about 1.5 times slower, more than about 2 times slower, more than about 3 times slower, more than about 5 times slower, more than about 10 times slower, more than about 20 times slower, more than about 30 times slower, more than about 50 times slower, more than about 100 times slower, more than about 200 times slower, more than about 500 times slower, more than about 1000 times slower, or more than about 5000 times slower than the noncoated composition/system).

Accordingly, in some aspects, disclosed herein is a system for introducing an agent into a cell, comprising:
a composition comprising an agent and a metal organic framework (MOF), wherein the composition is coated with a polymer;
a nozzle configured to retain the composition therein; and
compressed gas comprising CO₂ retained within a chamber that is connected to the nozzle.

The system described herein can used for delivering an agent into a cell, wherein the agent selected from the group consisting of a nucleic acid, a protein, a lipid, a small molecule, a bacterium, and a virus. In some embodiments, the agent is a therapeutic agent, wherein the therapeutic agent is a nucleic acid, a protein, a lipid, or a small molecule. In some embodiments, the therapeutic agent is a cancer therapeutic agent. In some embodiments, the agent is an antigen of a vaccine. In some embodiments, the antigen is a nucleic acid, a protein, a lipid, a small molecule, a bacterium, or a virus. The agent disclosed herein can be encapsulated or embedded within the MOF and/or on the outer surface of the MOF.

Accordingly, in some aspects, disclosed herein is a system for introducing a therapeutic agent or vaccine that comprises an antigen into a cell, comprising:
a composition comprising an agent and a metal organic framework (MOF);
a nozzle configured to retain the composition therein; and
compressed gas comprising CO₂ retained within a chamber that is connected to the nozzle.

In some embodiments, the compressed gas is configured to propel the composition into the cell. In some embodiments, the cell is a plant cell or a mammalian cell. In some embodiments, the cell is a plant cell. In some embodiments, the cell is a mammalian cell. In some embodiments, the cell is a human cell. In some embodiments, the mammalian cell is a skin cell. In some embodiments, the mammalian cell is a tumor cell. In some embodiments, the mammalian cell is a cell line.

The system described herein can comprises a cancer therapeutic agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (RolapitantHydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab).

A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, or eye.

In some aspects, disclosed herein is a method of treating a cancer comprising introducing a therapeutically effective amount of a cancer therapeutic agent into a cancer cell using the system disclosed herein, wherein the system comprises:
a composition comprising an agent and a metal organic framework (MOF);
a nozzle configured to retain the composition therein; and
compressed gas comprising CO₂ retained within a chamber that is connected to the nozzle.

In some embodiments, the cancer is skin cancer.

In some embodiments, a pressure for propelling the composition through the nozzle ranges from about 100 PSI to about 700 PSI. In some embodiments, a pressure for propelling the composition through the nozzle is about 500 PSI.

In some aspects, disclosed herein is a method of preventing a disease comprising introducing a therapeutically effective amount of an antigen into a cell (e.g., a skin cell) using the system disclosed herein, wherein the system comprises:
a composition comprising an antigen and a metal organic framework (MOF);
a nozzle configured to retain the composition therein; and
compressed gas comprising CO₂ retained within a chamber that is connected to the nozzle.

In some embodiments, the disease is an infection (e.g., a bacterial infection, a viral infection, or a yeast infection) or a cancer. In some embodiments, the antigen is a whole pathogen (e.g., a whole bacterium or a whole virus). In some embodiments, the antigen is a toxin/protein/lipid/polysaccharide of a pathogen (e.g., a bacterium or a virus). In some embodiments, the antigen is a tumor-associated antigen (TAA), a tumor-specific antigen (TSA), or a neoantigen, a viral-original “non-self” antigen.

In some embodiments, the therapeutically effective amount varies from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

As the timing of a cancer or metastatic condition can often not be predicted, it should be understood the disclosed methods of treating, preventing, reducing, and/or inhibiting a cancer or metastatic condition, or the use of any of the disclosed compositions or combinations for such treating, preventing, reducing, and/or inhibiting of a cancer or metastatic condition can be practiced prior to or following the onset of the cancer or metastatic condition to treat, prevent, inhibit, and/or reduce the cancer or metastatic condition. In one aspect, the disclosed methods or uses can be employed 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to a cancer or a metastatic condition; concurrently with the cancer or metastatic condition; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4,5, 6, 7, 8, 9, 10, 11, 12, 13, 14, 15, 16, 17, 18, 19, 20, 21, 22, 23, 24, 25, 26, 27, 28, 29, 30, 45, 60, 90 days, 4, 5, 6, 7, 9, 10, 11, 12 months, 2, 3, 4, 5, 6, 7, 8, 9, 10, 11, 12, 13, 14, 15, 16, 17, 18, 19, 20, 30, 40, 50 years or more post cancer or metastatic condition.

Dosing frequency for the nanoparticle or the nanogel drug composition disclosed herein, includes, but is not limited to, at least once every 12 months, once every 11 months, once every months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for the nanoparticle or the nanogel drug composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

In some aspects, disclosed herein is a method of introducing an agent into a cell comprising introducing the agent into the cell using the system described herein, wherein the system comprises:
a composition comprising an agent and a metal organic framework (MOF);
a nozzle configured to retain the composition therein; and
compressed gas comprising CO₂ retained within a chamber that is connected to the nozzle.

In some embodiments, the composition is comprised on a film. In some embodiments, the film is at the tip of the nozzle. In some embodiments, the film is a Parafilm®.

In some embodiments, the compressed gas is configured to propel the composition into the cell. In some embodiments, the cell is a plant cell or a mammalian cell.

In some embodiments, a pressure for propelling the composition through the nozzle ranges from about 100 PSI to about 700 PSI. In some embodiments, a pressure for propelling the composition through the nozzle is about 200 PSI or 500 PSI.

In some embodiments, the compressed gas further comprises nitrogen or an inert gas. In some embodiments, the inert gas is helium.

In some embodiments, the compressed gas further comprises air.

In some embodiments, the agent is selected from the group consisting of a nucleic acid, a protein, a lipid, and a small molecule. In some embodiments, the agent is a nucleic acid.

In some embodiments, the MOF is a Cu-based MOF, a Fe-based MOF, a Ni-based MOF, a Co-based MOF, a Zn-based MOF, or a Mg-based MOF. In some embodiments, the MOF is a zeolitic imidazolate framework (ZIF). In some embodiments, the MOF is ZIF-8.

In some embodiments, the agent is encapsulated within the MOF.

In some embodiments, the system of any preceding aspect further comprises a pharmaceutically acceptable polymer. In some embodiments, the polymer comprises one or more of polylactic acid (PLA), poly (lactic-co-glycolic acid) (PLGA), tannin, and a starch-based polymer.

In some aspects, disclosed herein is a method of introducing an agent into a cell, comprising:
providing a composition comprising the agent and a metal organic framework (MOF); and propelling the composition into the cell using compressed gas, wherein the compressed gas comprises CO₂.

In some aspects, disclosed herein is a method of introducing an agent into a plant cell, comprising:
providing a composition comprising the agent and a metal organic framework (MOF); and
propelling the composition into the cell using compressed gas, wherein the compressed gas comprises CO₂.

In some aspects, disclosed herein is a method of introducing an agent into a mammalian cell, comprising:
providing a composition comprising the agent and a metal organic framework (MOF); and
propelling the composition into the cell using compressed gas, wherein the compressed gas comprises CO₂.

In some embodiments, the composition is comprised on a film. In some embodiments, the film is at the tip of a nozzle.

In some embodiments, the compressed gas comprising CO₂ retained within a chamber that is connected to the nozzle.

In some embodiments, the compressed gas is configured to propel the composition into the cell. In some embodiments, the cell is a plant cell or a mammalian cell.

In some embodiments, a pressure for propelling the composition through the nozzle ranges from about 100 psi to about 700 psi. In some embodiments, the pressure for propelling the composition through the nozzle is about 200 PSI. In some embodiments, the pressure for propelling the composition through the nozzle is about 500 PSI.

In some embodiments, the compressed gas further comprises nitrogen or an inert gas. In some embodiments, the inert gas is helium.

In some embodiments, the compressed gas further comprises air.

In some embodiments, the agent is selected from the group consisting of a nucleic acid, a protein, a lipid, and a small molecule.

In some embodiments, the MOF is a Cu-based MOF, a Fe-based MOF, a Ni-based MOF, a Co-based MOF, a Zn-based MOF, or a Mg-based MOF. In some embodiments, the MOF is a zeolitic imidazolate framework (ZIF). In some embodiments, the MOF is ZIF-8.

In some embodiments, the agent is encapsulated within the MOF.

In some embodiments, the system of any preceding aspect further comprises a pharmaceutically acceptable polymer.

In some embodiments, the composition (for example, a composition in liquid form or solid form) is comprised within a film. In some embodiments, the film is at the tip of the nozzle. In some embodiments, the design of the nozzle with the film is as shown in FIG. 3 and/or FIG. 12.

In some embodiments, the system is as shown in FIG. 6.

The systems or methods described herein can increase the amount of the introduced agent in the target cell compared to a reference control (e.g., an amount of the agent detected in a subject in general or an amount of the agent in an untreated cell).

In some aspects, disclosed herein is a method for inserting genes into a plant cell using a gas activated delivery vehicle.

In some aspects, disclosed herein is a pharmaceutical composition comprising:
a therapeutic agent (e.g., DNA, protein, or a small molecule therapeutic);
a metal-organic framework (MOF) or a coordination polymer; and
wherein the therapeutic agent is encapsulated within the metal-organic framework or coordination polymer to form an encapsulated therapeutic agent, and
wherein the MOF is configured to dissolve at a pH not greater than 7.0.

In some embodiments, the MOF is configured to retain CO₂ within pores of the MOF.

In some aspects, disclosed herein is a system for administering a therapeutic agent, comprising:
a pharmaceutical composition comprising:
   a therapeutic agent (e.g., DNA, protein, or a small molecule therapeutic); and
   a metal-organic framework (MOF) or a coordination polymer;
a nozzle configured to retain the composition therein;
compressed gas comprising CO₂, communicatively connected to the nozzle and configured to propel the composition through the nozzle and to inject the composition into a target site.

In some embodiments, the pressure for propelling the composition though the nozzle ranges from 100 PSI to 700 PSI. In some embodiments, the pressure for propelling the composition through the nozzle is about 200 PSI or about 500 PSI.

In some embodiments, the compressed gas includes a ratio of CO₂ to an inert gas/nitrogen, the ratio configured to provide a desired acidic environment to the pharmaceutical composition upon delivering to the target site.

In some aspects, disclosed herein is a method of treating and/or preventing a disease or disorder in a subject in need thereof, comprising administering to the subject an effective amount of a pharmaceutical composition as disclosed herein, wherein the administration includes using the system disclose herein (e.g., a MOF Gun and a compressed gas comprising CO₂).

In some embodiments, the administration includes ZIF-8 as a carrier for the composition. In some aspects, disclosed herein is system using a metal-coordination polymer gene gun (MOF-Gun) which can include the following: pressurized gun chamber, controlling device, and material delivery system (gun nozzle).

In some embodiments, the gene gun can be operated using different gas sources. In some embodiments, the compressed gas comprises carbon dioxide, and optionally compressed air. In some embodiments, the first end of the gas pipe is connected to the gas source and the second end of the gas pipe is attached to a valve which commands the delivery of the gas through control of the opening and closing of the valve through a control device.

In other embodiments, the MOF-Gun components and their functions are: a gas supply and connection mechanism wherein the gas supply attaches to a gun compartment that allows the gas to flow into the pressurized chamber through the connection mechanism; a control device (this component is attached to the connection mechanism and allows for the control of an opening that regulates gas supply); and a material delivery system comprising a gun nozzle wherein the microcarrier holders are placed above a stopping screen. The screen serves as a gating system that regulates particle diffusion based on size. This can prevent large particles from going through and minimizes cellular damage. The gating screen is interchangeable to allow for larger or smaller particles.

In some embodiments, the MOF-Gun has been used to deliver the following materials: a plurality of nucleic acids encapsulated in a metal coordination polymer that contains zinc, or another metal, connected by methyl-imidazole, or another organic ligand, in a coordination framework that fully encapsulates the genetic material. In some embodiments, the zeolitic imidazolate framework-eight, ZIF-8, is used as a representative material type. Other polymorphs or crystal systems of zeolitic imidazolate can also be used including, but not limited to, ZIF-L, ZIF-C, dia(Zn).

In some embodiments, the particle can be synthesized in sizes ranging from 300 nm to greater than 1 μm. In some embodiments, the particle can be synthesized in a dry powdered form.

In some embodiments, the material delivery system comprises a particle loading gun nozzle wherein the ZIF-8 encapsulated genetic material is placed on top of a sterilized parafilm barrier placed in between two sterilized metal washers.

In some embodiments, the ZIF-8 is loaded as a suspension in water, or buffer, or a dry powdery form.

In some embodiments, a biodegradable metal coordination polymer is used that contains zinc, or another metal, connected by methyl-imidazole, or another organic ligand, in a coordination framework that fully encapsulates the genetic ZIF-8 which is used to encapsulate the genetic material inside its porous structure. This gene carrier allows for biolistic gene delivery as a material that can control and release the genetic payload in a gene transfection process by reacting with the carrier gas.

In some embodiments, the metal coordination polymer is able to release its cargo via reaction with the carrier gas. For example, carbon dioxide is chemically converted into carbonic acid, which causes the degradation of ZIF-8 and the release of the genetic cargo.

In some embodiments, the metal coordination polymer that contains zinc, or another metal, connected by methyl-imidazole, or another organic ligand, in a coordination framework like ZIF-8 is also used as the microcarrier delivery system to deliver genes by protecting them inside its porous structure such that it would otherwise degrade or decompose in cellular media through the gene delivery process.

In some embodiments, ZIF-8 is used as a cost effective, biodegradable, biocompatible and efficient gene carrier with a higher genetic payload compared to the heavy metal gene carriers of tungsten and gold that have been previously used in biolistic gene delivery. In some embodiments, ZIF-8 is used as a gene carrier with a tunable pore structure, pore size, and particle size that can be synthesized under mild and biocompatible reaction conditions. In some embodiments, ZIF-8 is used as a novel carrier with a stable structure resistant to nucleases and temperature stressors that would otherwise degrade the genetic material coated outside the heavy metal carriers.

In some embodiments, the amount of genetic material to be delivered can be controlled and calculated with ZIF-8 encapsulation process and can be more precise than the surface coating of the genetic material on outside of the heavy metal particles.

In some embodiments, the genetic material encapsulated in ZIF-8 can be stored at standard temperature (for example, room temperature) and standard pressure conditions without using refrigeration.

### EXAMPLES

The following examples are set forth below to illustrate the compositions, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Carrier Gas Triggered Biolistic Delivery of Agents

Provided herein is a novel method for delivery of agents into a cell. In this example, Zeolitic Imidazolate Framework-8 (ZIF-8) is used as an efficient and stable gene carrier that can be used with a low-cost gas-powered “MOF-Gun” for direct delivery into cell (for example, a plant tissue). ZIF-8 is more economical, biodegradable, and affords protection to the DNA against degradation compared to metal nanoparticles. By using CO₂ as a reactive carrier gas, a rapid local pH drop in planta occurs, thus dissolving the ZIF-8, and subsequently releasing the DNA.

Changes in the earth's climate and population are placing strains on agricultural production, as extremes in temperature, precipitation, and population growth have made producing crops more of an economical risk. Plant genetic engineering has improved nutritional quality and yields by promoting resistance to herbicides, pesticides, insects, diseases, and drought. “Germ line” genetic modification used to create these genetically modified organisms has radically improved access to high quality food but has been met with opposition from governments, consumers, and farmers. An alternative is to transiently deliver genes to crops in a way that does not alter the plant's genome. The most widely used method for transient plant genetic engineering is the “biolistic method” that uses so-called gene guns to physically deliver DNA into plant cells and can be used for large scale crop production. For gene expression using a biolistic method, nucleic acid molecules are adsorbed onto heavy metal particles, such as gold or tungsten, and then accelerated to high speeds via pressurized gas flow—typically helium—and bombarded directly into the targeted tissue. This allows genetic material to directly enter the cytoplasm, bypass cellular endosomes, and avoid complex endosomal escape processes. This simplicity has allowed biolistic methods to make steady progress over traditional methods like direct DNA injection and Agrobacterium-mediated gene transfection. While this technique is quite useful, it has several drawbacks. The Helios gene gun used for biolistic particle delivery and the materials needed for sample preparation (e.g., gold or tungsten) make this approach quite expensive, with costs exceeding US$50,000. Loading the particles with DNA is a cumbersome task, requiring its own apparatus and drying of particles. Further, the metal microparticles are non-biodegradable, which means they will persist in the plants indefinitely. Finally, once the genetic material is loaded onto the metal microparticles, it is susceptible to nuclease degradation, and so these “biolistic bullets” have to be refrigerated (4° C.) and still have a limited shelf life of only 2-3 months after preparation.

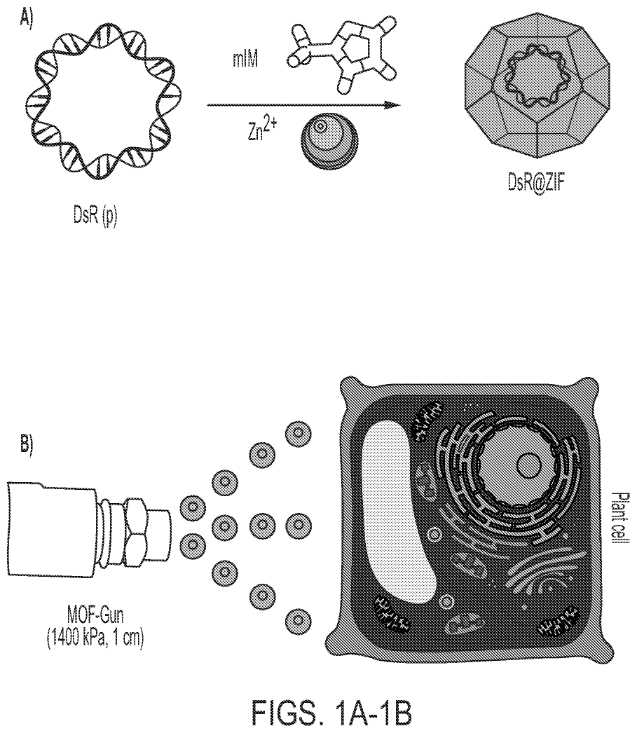

Recent developments in the biomimetic mineralization of biomacromolecules with the metal-organic framework (MOF) ZIF-8 allowed the investigation of a new system and method for gene delivery. MOFs are a class of hybrid porous materials made from interconnected metal nodes and organic linkers and have proven to be excellent candidates for gas separation, storage, and chemical catalysis. ZIF-8, a type of MOF composed of the organic ligand methylimidazole (mIM) interconnected by Zn²⁺ nodes, nucleates on the surface of biomacromolecules via a “biomimetic” mechanism fully encapsulating them (FIG. 1A). Biomolecules including enzymes, genetic material, bacteria and yeast, whole viruses, and intact liposomes have been encapsulated and shown enhanced stability against heat and enzyme degradation. The rigidity of the MOF structure enhances the thermal stability of the encapsulated biomaterial by inhibiting the necessary thermal motion that leads to unfolding and serves as a physical barrier against proteases and nucleases. ZIF-8 biodegrades slowly, which has made it an attractive material for vaccine and drug delivery applications, where it can break down releasing the encapsulated biomaterial into tissues over the course of many days.

The low toxicity of the ZIF family of MOFs, which biodegrades back into their discrete zinc metal ions and imidazolate linkers, has made them a useful biocompatible protector for highly sensitive therapeutic biomolecules. ZIF particle size can be controlled by altering the concentrations of the starting biomacromolecule, zinc, and imidazole solutions yielding particles ranging in size between several nanometers to micrometers. These formulations have been used to deliver DNA and mRNA into mammalian cells by endocytosis. Endosomes acidify and dissolve the ZIF, releasing the biomacromolecules. Plant cells do not endocytose nanomaterials and must be physically pushed into the cell. To do that, a novel, low-cost particle delivery system called a “MOF-Gun” (FIG. 1B) was designed to deliver the biomimetically mineralized genetic material into the target tissue directly.

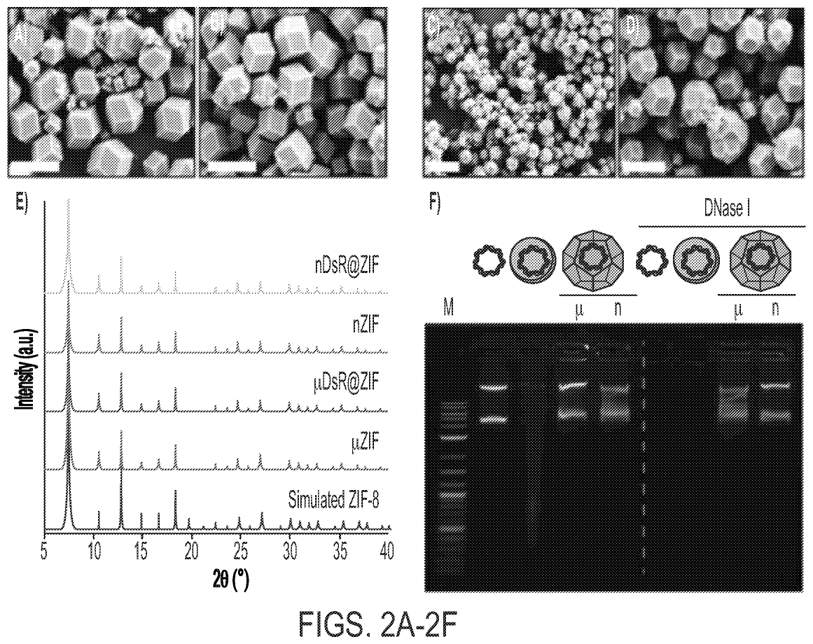

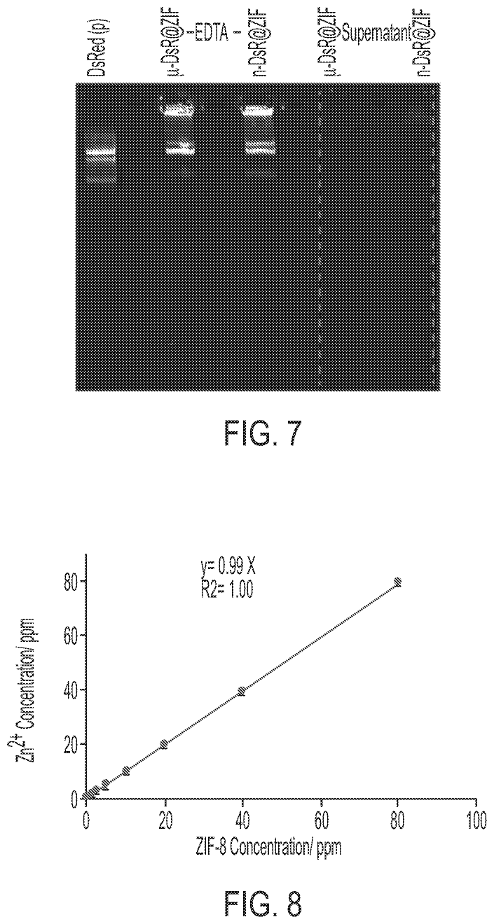

To establish this approach, an experiment has been conducted that provides a fluorescent output. The pEGB35S:DsRed DNA plasmid was selected, which encodes for a red fluorescent protein derived by Discosoma sp. (FIG. 6). The synthetic conditions needed to quantitatively encapsulate the DsRed plasmid within ZIF-8 to form DsR@ZIF was then systematically identified. DNA transfection requires very little material (2.5 μg of DNA per 1.0 mg of ZIF-8) and the metal to ligand ratio were optimized to produce both micro (1.02±0.03) μm) and nano (355.00±48.38) sized crystals of pristine ZIF-8 (FIGS. 2A and 2C respectively) and DNA loaded ZIF-8 (FIG. 2B and FIG. 2D). The composites were analyzed by powder X-ray diffraction (PXRD), which confirmed the sodalite ZIF-8 topology (FIG. 2E). Scanning electron microscopy (SEM) shows uniform rhombic dodecahedral DsR@ZIF crystals and confirms that the encapsulation of the DsRed plasmid does not affect the crystal morphology (FIG. 2D). Characterization with thermo gravimetric analysis (TGA) and N2 adsorption data show minimal differences between pristine ZIF-8 and DsR@ZIF (FIGS. 7A and 7B). The EDAX (FIGS. 7C-7F) data supports DNA encapsulation, as indicated by faint signals for the presence of phosphorus in DsR@ZIF.

The protection of genetic materials against nuclease degradation is an important aspect of efficient gene transfection. The stability of the encapsulated DNA against nuclease degradation was investigated in the nano and micro DsR@ZIF formulations and compared to pristine DsRed plasmid as well as DsRed coated on tungsten metal microparticles (DsR@W). The samples were incubated with DNase I for 10 mins. The DsR@ZIF formulations were removed from solution and the ZIF shell was dissolved in 0.5 M ethylenediaminetetraacetic acid (EDTA). The solutions were run on an agarose gel, as shown in FIG. 2F. The left of the agarose gel shows the electrophoretic mobility of the DNA plasmid without any nuclease treatment. On the right of the gel, the bands corresponding to free plasmid and DsR@W are notably absent, indicative of DNA digestion. On the other hand, the DsR@ZIF formulations still contain DNA, showing they prevented nuclease degradation. In other words, the DNA is on the inside of the ZIF-8, and therefore inaccessible to nucleases.

After confirming that the DsRed plasmid is encapsulated and protected in ZIF-8, the gene transfection process was investigated. This MOF-Gun (FIG. 3A) was modified from a previously disclosed design with specific modifications to the firing tip (FIG. 3B) to allow for firing dry MOF powder-a thin piece of parafilm sandwiched between two metal washers (FIG. 3C) and seated inside the tip. ZIF powder is set onto the top of this thin parafilm membrane and resides there until fired. The biolistic delivery parameters were then optimized (operating pressure and distance between the gun nozzle and the targeted tissue) for the gun by firing ZIF-8 particles loaded with the fluorescent dye Cy-5 into home-made ballistic gel, as schematically illustrated in FIG. 3E. Agarose gel (2%, 2 cm×2 cm) emulates soft tissues in the body and allows for the imaging of penetration via fluorescence microscopy, which helps calculate the distance travelled by the fluorescently labeled ZIF-8 particles (FIG. 3F). It was found that using the MOF-Gun at a 1400 kPa and 1 cm away from the target, the ZIF-8 particles were principally lodged within the first 0.3 mm of the gel with several particles traveling as far as 3 mm from the gel surface (Table 1). Importantly, the firing of the MOF gun did not damage the gel surface as shown in images before firing (FIGS. 3F and 3G) and after firing (FIGS. 3H and 3I). Based on these results, the pressure of 200 PSI and the tip-to-sample distance of 1 cm was selected for the remaining experiments.

TABLE 1
Shooting Parameters- Plant

| | Penetration depth under | | |
|---|---|---|---|
| Distance from | different pressures/mm | | |
| the nozzle/cm | 700 kPa | 1400 kPa | 2100 kPa |
| 1 | 0.5 | 3 | X |
| 2 | 0.4 | 2.5 | 1.5 |
| 3 | 0.35 | 1 | 1.4 |
| 4 | 0.6 | 1.5 | 1.7 |

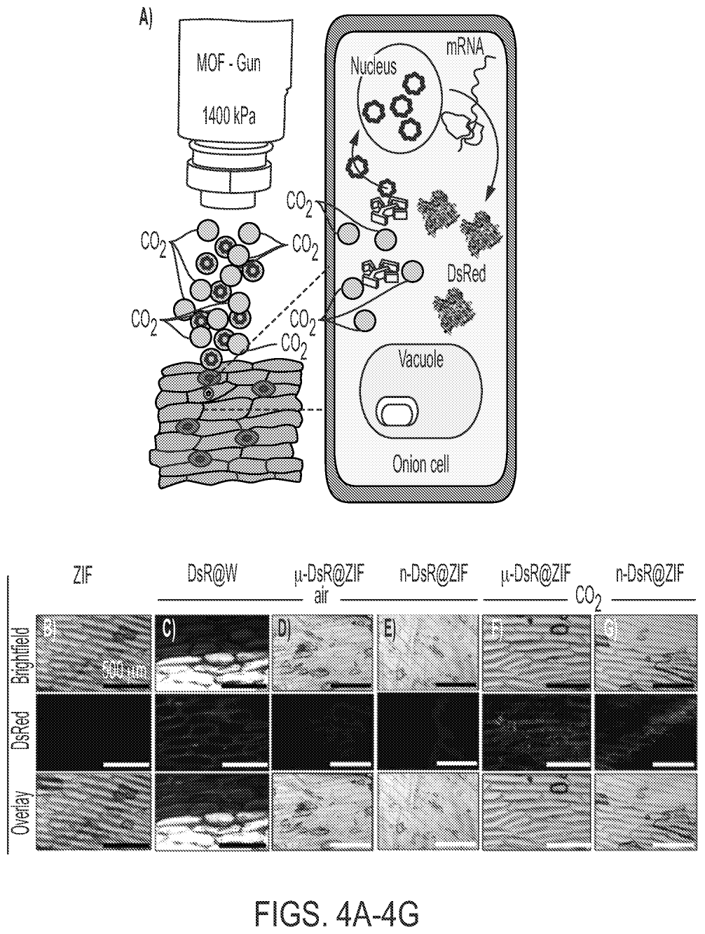

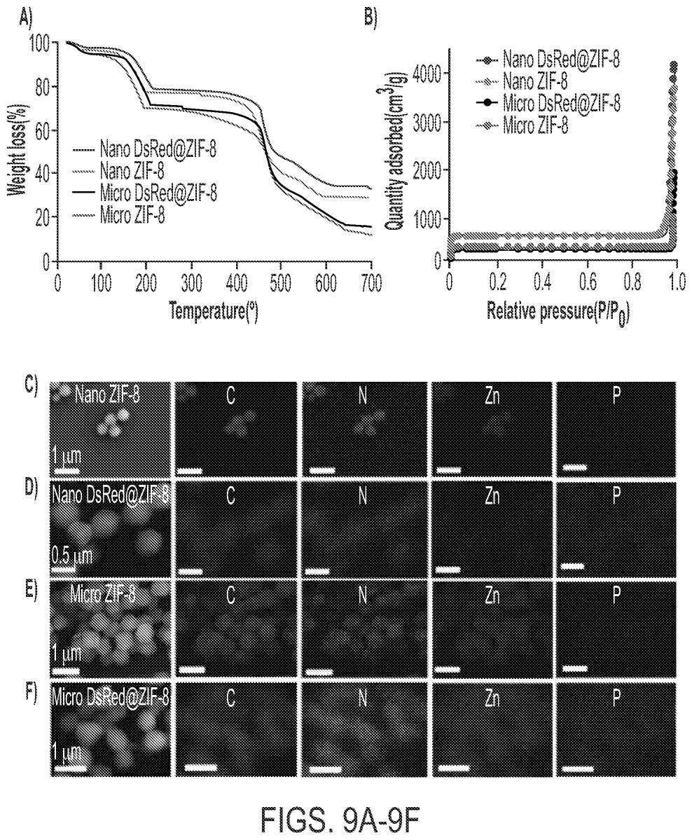

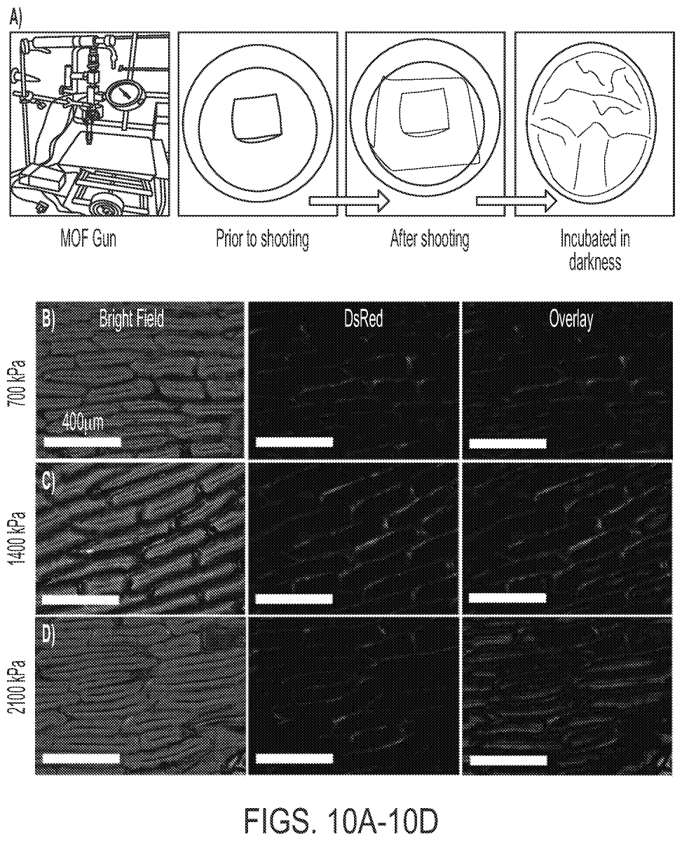

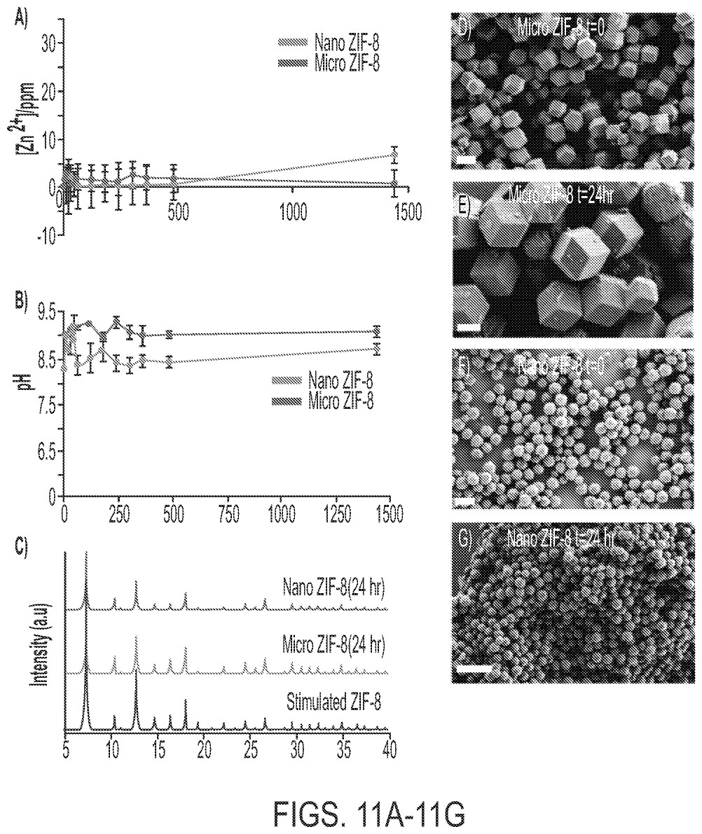

Transfection was done using white onions as the test plant as have large cells with well-defined structures. Indeed, they were the plant tested in the original biolistic gene delivery paper. For gene expression to occur, DsR@ZIF must enter the cell, the ZIF shell must dissolve, and then the genetic payload must go to the nucleus (FIG. 4A). The DsRed plasmid encodes mRNA for the synthesis of a red fluorescence protein, which can be monitored through confocal laser scanning microscopy (CLSM). In the first experiment, DsR@ZIF, DsR@W (positive control), and pristine ZIF-8 (negative control) were delivered using compressed air as the propellant (200 PSI, 1 cm) into an onion bulb, which was then incubated in the dark for 24 h (FIG. 9A). The negative ZIF control produced no fluorescence while the DsR@W produced fluorescence (FIGS. 4A, 4B respectively). There was little fluorescence and thus low gene transfection in either the micro- or nano-particle formulations of DsR@ZIF. Since DsR@W induced fluorescence, the gun was working as intended. It was possible the ZIF shell was not dissolving fast enough to release the DNA allowing for efficient transfection. CO₂ has been shown to acidify the interior of MOFs and ZIF-8 is capable of adsorbing CO₂ and dissolves at low pH, so using CO₂ as the carrier gas can promote transcription by dissolving the ZIF shell. Indeed, using CO₂, gene expression was observed from both nano and micro DsR@ZIF formulations (FIGS. 4C, 4D) that matched or exceeded the positive DsR@W control.

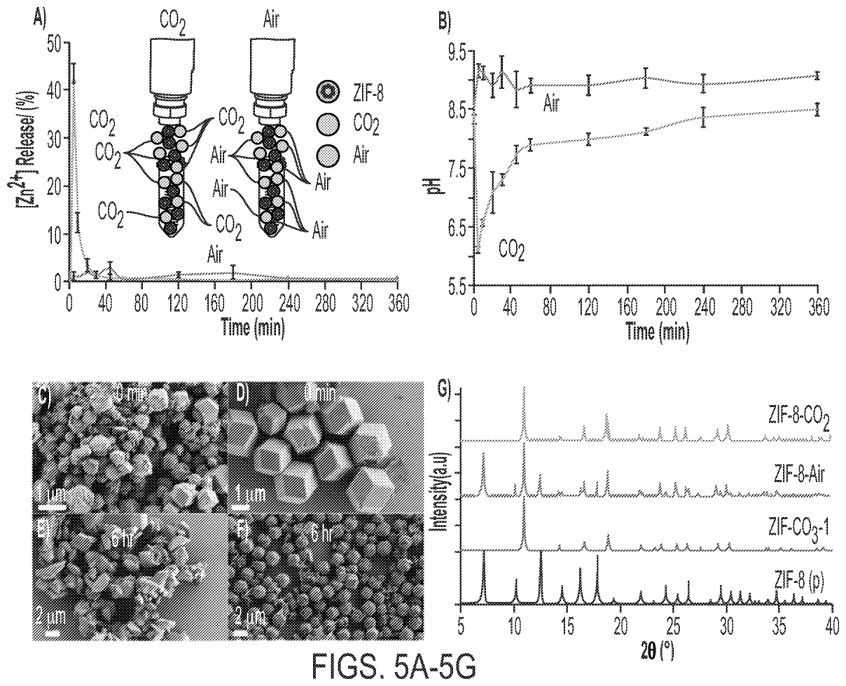

To test if the improved gene transfection process with CO₂ was a result in the acidification, a study was performed using pristine ZIF-8 crystals, as illustrated in the insert of FIG. 5A. In this experiment, ZIF-8 was shot into water using either CO₂ or compressed air and time resolved measurements of free Zn²⁺ as well as pH were done. Initially onion extract was used but the extract was very viscous and sticky, which made it difficult to get accurate pH readings. In brief, 25 mg of pristine ZIF-8 was shot into a tube containing water (initial pH 8.76) with either CO₂ or compressed air. After shooting ZIF-8 into the water, the Zn²⁺ concentration was monitored in time intervals for up to 6 h using inductively coupled plasma mass spectroscopy (ICP-MS) and the pH of the solution was monitored using a micro-pH probe connected to a customized controller. As shown in FIG. 5A, when using CO₂ as the carrier gas, there is a huge increase of free Zn²⁺ in the solution peaking at 10 mins post injection. This peak alone accounts for nearly 45% of the original Zn²⁺ in the ZIF-8 used. There was no such burst release when compressed air was used as the carrier gas. After about 10 mins, the Zn²⁺ concentration in the solution gradually decreased, indicating Zn²⁺ was leaving the solution. Correspondingly, at the time the Zn²⁺ concentration was at its highest, the solution pH was at its lowest (6.0-6.2) and then gradually returned to its starting pH over the next 6 h whereas the pH of the solution of ZIF-8 delivered with compressed air did not show significant changes (FIG. 5B). This difference in pH changes between carrier gas can be explained by CO₂ dissolution in water making weakly acidic carbonic acid, which yields CO₃²⁻ and HCO₃⁻ anions. Low pH causes dissolution and release of the encapsulated biomolecules by decomposition of the ZIF-8 matrix. The increase of the pH after 10 mins can be explained by the gradual decarbonation of the water as the CO₂ reenters the air from the water. SEM data obtained from samples of ZIF isolated several minutes after shooting them into water with CO₂ (FIG. 5C) shows clear surface changes compared to the ZIF-8 shot with compressed air (FIG. 5D), which still looks pristine. Samples shot with CO₂ and allowed to incubate in solution for 6 h show totally different morphologies (FIG. 5E) whereas the ZIF-8 shot with air show only minor changes to the crystals (FIG. 5F). The PXRD data (FIG. 4G) obtained from the recovered powder following delivery with CO₂ or compressed air both show prominent new reflections, a particularly large one at 11° 2Θ in both samples, which corresponds to the previously reported ZIF polymorph ZIF-CO₃-1 (also reported as ZIF-C). When CO₂ was used, pure ZIF-CO₃-1 is formed; however, when air was used, a significant fraction of the original ZIF-8 remained. Carbonated water substantially increases the CO₂ adsorption of ZIF-8, but affects its crystal structure. This phenomenon has been explained based on an irreversible two-step reaction that happens when ZIF-8 and carbonated water mix, initially creating zinc carbonate and 2-methylimidazole crystals, which leads to the dissolution of the ZIF-8 structure. Therefore, the lower gene transfection efficiency when air was used can be attributed to the fact that, without the help of CO₂, the ZIF-8 does not dissolve or degrade quickly and the encapsulated DNA is therefore transcribed slowly. This slower rate of transcription may ultimately be useful as the “time delayed” aspect of ZIF-8 dissolution has recently been used to promote better immune responses to bacterial infections in vivo and it might be applicable to gene transfection in vivo and in planta as well.

In summary, this example has demonstrated an efficient method to utilize ZIF-8 as a biocompatible, cost-effective metal organic framework to carry and protect DNA plasmids in biolistic applications. Qualitatively, ZIF-8 performs as well as tungsten microparticles in the transfection of onion cells following gene gun delivery. This study has further shown that the transfection efficiency of DNA plasmids using ZIF-8 can be controlled by changing the composition of the carrier gas. When neat CO₂ is used, the ZIF carrier brings with it all that is necessary for its fast dissolution and rapid transfection. This allows for controllable biolistic delivery in a broad range of organisms.

### Example 2. Methods and Materials

### Materials.

All reagents here described were used without further purification. Ethylenediaminetetraacetic acid (EDTA), 2-methylimidazole, zinc acetate dihydrate were purchased from Sigma-Aldrich. pEGB 35S:DsRed:Tnos (GB0361) was gifted from Diego Orzaez (Addgene plasmid ##68220; n2t.net/addgene:68220; RRID:Addgene_68220) was purchased from Addgene (USA). Propidium iodide and nuclease free water was purchased from Thermo Fisher Scientific (Waltham, MA, USA). DNase I and SYBR Gold DNA stain were purchased from New England Biolabs. Ultrapure water was obtained from ELGA PURELAB flex 2 system with resistivity measured to at least 18.2 MΩ-cm. White onions were purchased from the local grocery (Walmart). PureYield™ Plasmid Maxiprep System for plasmid purification and extraction was purchased from Promega (USA).

### MOF-Gun Schematic.

The MOF gun is comprised of the components as shown in FIG. 6

### MOF-Gun Components in Detail.

Gas is delivered to the device from either a gas canister (Maddog Heavy Duty Paintball Tank Remote Coil connected to a tank, as shown in FIG. 6) or 12 g CO₂ adapter (AMC Quick Charge 12 g CO₂ Adapter—not shown) either of which is attached to the Universal Fill adapter (Ninja Paintball Universal Fill Adapter). Gas is from food grade 12 g CO₂ cartridges or compressed air (oil-free membrane compressor with built in water-oil filtration).

Gas enters by opening the needle valve, which pressurizes the system. A large analogue pressure gauge (reading both SI and Imperial Units) indicates pressure. Once desired pressure is reached, the needle valve is closed. The firing components of the MOFGun are composed of the following parts, assembled per FIG. 6. A high-pressure solenoid (Redhat Mfr #826H200) suitable for corrosive and inert liquid/gas applications with a maximum operating pressure of 3500 kPa is controlled via an adjustable relay (Dayton Time Delay Relay Mfr #6A855) interfaced to a Square D Relay Socket (Mfr #8501NR61) standard, octal, 11 pin configuration. The relay/solenoid is activated by a connected miniature firing push button.

Metal pipes and connectors are stainless steel rated at or above 3500 kPa and purchased in the United States from McMASTERCARR. All components are designed and measured to imperial units (US standards). Thread and thread sizes of ¼ inch NPT. The tip is a prefabricated Solid-Stream Spray Nozzle manufactured by McMASTER CARR (Mfr #7611T53) Washer inserts have approximately 0.325 cm inner diameter and 0.685 cm outer diameter and fit precisely within the tip.

### Loading and Firing Procedures.

To make the loading washers, a small piece of parafilm is sandwiched between two washers and then stretched to form a thin membrane. The loading washers then was lodged into the gun nozzle. Then, 2.50 mg of powdered/solid sample of DsR@ZIF (5 μg of DsRed) was weighed and loaded onto the parafilm membrane. After that, the gun nozzle was screwed back into the MOF-Gun and pressure and firing distance were adjusted. The relay timer was set to 0.9 s and the firing button was pushed, releasing the particles. This can eject ZIF-8 at speeds sufficient to penetrate the skin. These experiments should be conducted with safety classes, gloves, and lab coat. Discharging ZIF-Gun in a fume cabinet with airflow is recommended.

### Time Resolved pH Meter.

A time resolved pH meter was constructed using a pH/ORP adapter (Phidgets.com) connected to a Phidget VINT Hub. The Hub interfaces to a PC. The following script was used to generate data:

```
#Add Phidgets library
from Phidget22.Phidget import *
from Phidget22.Devices.VoltageInput import *
#Required for sleep statement
import time
#Create
ch = VoltageInput( )
#Set addressing parameters to specify which channel to open (if any)
ch.setIsHubPortDevice(True)
ch.setHubPort(0)
#Open
ch.openWaitForAttachment(5000)
#Set the sensor type to match the analog sensor you are using after
opening the Phidget
ch.setSensorType(VoltageSensorType.SENSOR_TYPE_1130_PH)
#Record data points
count = 0
#define a variable
sensorValue = ch.getSensorValue( )
#Use your Phidgets
while (True):
  #Update user
  print(“Logging data...”)
  #Write data to file in CSV format
  with open (‘phidgets_temperature.csv’,‘a’) as datafile:
  datafile.write(str(sensorValue) + “\n”)
  #Increment count
  count += 1
  #If 10 data points have been recorded, close file and exit program
  if(count == 10):
  print(“Logging complete, exiting program”)
  exit( )
  time.sleep(0.5)
main( )
```

### DsRed Plasmid Purification.

Kanamycin supplied LB agar plates were streaked with pEGB 35S:DsRed transformed Escherichia coli (purchased from Addgene), using a sterilized loop and incubated overnight at 37° C. Single colonies were added to 5 mL of LB kanamycin-containing media and incubated overnight at 37° C. with continuous shaking. Further, the cells were amplified in 1000 mL of LB media, incubated at 37° C. for 19 h, and their optical density monitored. At an optical density value of 0.9, cells were harvested by centrifugation at 5000×g for 10 mins, and the plasmid isolated using a Promega Maxiprep kit. Steps for plasmid isolation and purification are described elsewhere. Purified pEGB 35S:DsRed was collected in DNase-free water and stored in −20° C. for further use. Concentration of the DNA was determined by NanoDrop™ UV-Vis spectroscopy.

Synthesis of pEGB 35S:DsRed@ZIF-8 Micro and Nano Composites.

TABLE 2
Ingredients used for synthesis of micro ZIF-8 formulations.

| | | 1M zinc | Nuclease- | DsRed |
|---|---|---|---|---|
| | 3M mlM | acetate | free water | (p) |
| | volume | volume | volume | volume |
| Formulation | (mL) | (mL) | (mL) | (mL) |
| Micro ZIF-8 | 0.213 | 0.010 | 0.777 | 0 |
| Micro DsR@ZIF | 0.213 | 0.010 | 0.752 | 0.025 |

TABLE 3
Ingredients used for synthesis of nano ZIF-8 formulations.

| | mIM | Zinc | Nuclease | DsRed (p) |
|---|---|---|---|---|
| | weight | acetate | free water | volume |
| Formulation | (mg) | weight (mg) | volume (mL) | (mL) |
| Nano ZIF-8 | 95.0 | 7.0 | 1.000 | 0 |
| Nano DsR@ZIF | 95.0 | 7.0 | 0.752 | 0.025 |

Two formulations were used for the encapsulation of DsRed plasmid. The micro-DNA-containing ZIF formulation was prepared as follows: 25 μL of 1 mg/mL sDsRed plasmid were added to a solution of 752 μL of nuclease-free water and 213 μL of 3M 2-methylimidazole. Then, 10 μL of 1 M zinc acetate dihydrate were added. The resulting solution was mixed thoroughly, and almost immediately following the addition of zinc, the mixture went from colorless to cloudy. The reaction proceeded for 18 h at RT, and the DNA@ZIF crystals harvested by centrifugation 10,000×g for 15 min. The resulting supernatant was collected and used for encapsulation efficiency determination. Excess precursors were removed by two additional water washes, and crystals were allowed to dry at RT for used as is. Similarly, the micro pristine ZIF-8 solution was prepared by mixing 777 μL of nuclease-free water, 213 μL of 3M 2-methylimidazole, and 10 μL of 1 M zinc acetate dihydrate. The nano formulation was prepared following a protocol described elsewhere. Briefly, 95 mg of 2-methylimidazole and 7 mg of zinc acetate dihydrate were dissolved in 500 μL of nuclease-free water separately. 25 μL of 1 mg/mL DsRed plasmid was added to the 2-methylimidazole solution. Next, the zinc solution was added and the solution thoroughly mixed. The encapsulation proceeded for 15 min at RT, and the DNA loaded ZIF-8 crystals harvested by centrifugation 10,000×g for 15 min. The supernatant was collected and used for assessment of encapsulation efficiency. Accordingly, the nano pristine ZIF-8 crystals were afforded by reacting 95 mg of 2-methylimidazole and 7 mg of zinc acetate dihydrate in a final volume of 1000 μL of nuclease-free water, for 15 min at RT. Unreacted precursors were removed by water washes (2×) and dried in open air at RT.

### Synthesis of Cy5@ZIF.

Synthesis of Cy5@ZIF was done following the same procedure used to synthesize nano DsR@ZIF. Briefly, 95 mg of 2-methylimidazole and 7 mg of zinc acetate dihydrate were dissolved in 500 μL of water separately. Then, 25 μL of 1 mg/mL Cy5 stock was added to the 2-methylimidazole solution and mixed well. Once thoroughly mixed, zinc acetate dihydrate solution (500 μL) was added to Cy5, 2-methylimidazole mixture and mixed well. The mixture was allowed to react for 15 min at RT. Blue Cy5@ZIF powder was harvested by centrifugation at 17,000×g for 15 min. The Cy5@ZIF was washed twice with water and dried at RT.

### Fluorescence Spectroscopy.

Fluorescence spectroscopy was used to determine encapsulation efficiency. Briefly, propidium iodide (1 mg/mL) was added to the supernatant of the DsR@ZIF. A solution of 1 mg/mL pristine DNA was used as control. The reaction proceeded at RT for 10 min (covered in foil). Fluorescence spectra were then collected using a in a Tecan Spark 20M plate reader (Ex λmax=535 nm and Em λmax=617 nm). Data collected revealed an encapsulation efficiency of 95.5% for micron formulation and 94.8% for the nano formulation.

### Material Characterization.

Surface morphology was investigated using a Zeiss Supra 40 scanning electron microscope at 1 kV and a working distance of 6 mm. Surface area measurements were carried out on a Micrometrics ASAP 2020 surface area analyzer by nitrogen adsorption under 77 K. Surface area was independently analyzed using the Brunauer-Emmett-Teller (BET) method and pore size determined under the nonlocalized density functional theory with a carbon slit pore model 18. Sample activation took place before analysis. Briefly, all samples were soaked in MeOH and dried in high-vacuum overnight. The crystals were then soaked in dichloromethane overnight. Lastly, the dichloromethane was removed by high vacuum overnight. Sample degassing was done at 120° C. under vacuum for 12 h.

PXRD data was measured using a Rigaku SmartLab X-ray diffractometer equipped with CuKα (1.54060 Å) at 30 mA and 40 kV. All samples here reported were activated prior to analysis. Data for each individual PXRD collected was uploaded it into the Global fit software and analyzed from 5° to 40° (2θ).

Thermogravimetric analysis of each sample was done in a TA Instruments SDT Q600 Analyzer. The temperature was ramped up from 30 to 800° C., under an N2 atmosphere, under a constant heating rate of 5° C. min⁻¹.

### Agarose Gel Electrophoresis of DsR@ZIF-8.

As shown in FIG. 7, Agarose gel electrophoresis was used to confirm that the DNA is associated with DsR@ZIF. The centrifuged, washed and exfoliated DsR@ZIF pellet, supernatant and controlled DNA were loaded onto 1% agarose gel and was allowed to run at 70 V for 1 h.

### DNase I Assay.

DNase I treatment was used to evaluate the protection of DNA from nuclease degradation through encapsulation inside ZIF-8. The treatment was done using TURBO™ DNase (Invitrogen) following manufacturer's protocol. In brief, DNase I (1 μL of 2 U/μL) was added to both micro and nano DsR@ZIF, pristine DsRed plasmid DNA, and DsR@W. The reaction was allowed to occur for 10 mins at 37° C. Untreated pristine DsRed plasmid DNA, micro DsR@ZIF, nano DsR@ZIF and DsRed@W were used as controls. Next, 1 μL of 0.5 M EDTA was added to terminate the enzymatic reaction by denying it the divalent cations it needs for the degradation reaction. The treated DsR@ZIF samples were centrifuged, and the supernatants were removed. Finally, 100 μL of 0.5 M EDTA was added to dissolve the ZIF-8 crystals thereby releasing the encapsulated DNA. The solutions were run on an agarose gel. It was noted that the bands corresponding to free plasmid and DsR@W are notably absent, indicative of DNA digestion. On the other hand, the DsR@ZIF formulations still contain DNA, showing they prevented nuclease degradation. In other words, the DNA is on the inside of the ZIF-8, and therefore inaccessible to nucleases.

Optimization of shooting parameters of the particle delivery system. Cy-5 fluorescent dye labeled ZIF-8 was shot at different distances with different pressure values ranging from 100 PSI to 300 PSI to obtain the maximum penetration depth conditions. This process was carried out using 2×2 cm 2% agarose gels made using a silicon mold. 2% agarose gels were used for this optimization as it has previously been reported as a model for emulating soft tissue. After shooting fluorescently labeled ZIF-8 into the gel, the penetration depth of ZIF-8 particles inside of the gel was measured by epifluorescence microscopy using a microscope calibration slide (visible behind the gel in FIG. 3).

Biolistic Delivery of DsRed@ZIF-8 into Onion Tissues.

The synthesized micro and nano size crystals of DsRed@ZIF were dried at RT before being used for bombardment. The optimized bombardment parameters for the onion epidermis were 200 PSI, 1 cm distance between the gun nozzle and tissue. Bombardment of onion bulbs was performed according to the lab safety protocols inside a fume hood. After bombardment, the onion bulbs were placed inside a culture plate on a napkin wetted with DI water, following which the plates were covered with aluminum foil and incubated for 24 h. The epidermis tissue layer was then carefully excised from the bulb and imaged using confocal laser microscopy and epifluorescence microscopy to evaluate the DsRed fluorescence protein expression. Air and CO₂ gas were used as particle propellants, and both sets of data were analyzed for comparison.

Study of ZIF-8 Release Profile with CO₂ and Compressed Air.

25 mg of pristine ZIF-8 was shot directly into 10 mL of DI water using CO₂ and compressed air as particle propellants respectively. After the delivery of ZIF-8 into DI water, the falcon tubes were capped and allowed to properly mix on a rotisserie. 0.1 mL aliquots of the supernatant were collected at each time interval and replaced with DI water. pH of the solution at each time point was recorded using a micro-pH probe connected to a customized controller. To obtain Zn²⁺ concentrations, 9.9 mL of 2% HNO₃ acid was added to 0.1 mL of the supernatant and allowed to react for 48 h before ICP-MS measurements. The standard curve for Zn²⁺ was prepared by dissolving/digesting 25 mg of pristine ZIF-8 in 10 mL of 2% HNO₃ acid. Both experiments were done in triplicate (n=3). (FIG. 8).

Study of ZIF-8 Release Profile without Shooting, Under Standard RT Conditions.

To study the ZIF-8 stability/releasing profile without shooting, 25 mg of pristine ZIF-8 in powder form was dispersed and resuspend in 10 mL of DI water in falcon tubes. Then the falcon tubes were capped and allowed to properly mix in a rotisserie. 0.1 mL aliquots of the supernatants were collected at each time interval and replaced the solution with DI water. pH of the solution at each time point was also recorded using a micro-pH probe connected to a customized controller. Then, 9.9 mL of 2% HNO₃ acid was added to 0.1 mL of the supernatant and allowed to digest for 48 h before ICP-MS measurements of Zn²⁺. Standard curve for Zn²⁺ was prepared by dissolving/digesting 25 mg of pristine ZIF-8 in 10 mL of 2% HNO₃ acid. Both experiments were done in triplicate (n=3).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of introducing an agent into a cell, comprising:
providing a composition comprising the agent and a metal organic framework (MOF); and
propelling the composition into the cell using compressed gas, wherein the compressed gas comprises CO₂.

2. The method of claim 1, wherein the composition is comprised on a film.

3. The method of claim 1, wherein the composition is retained within a nozzle.

4. The method of claim 3, wherein the film is at the tip of the nozzle.

5. The method of claim 1, wherein the gas is retained within a chamber.

6. The method of claim 5, wherein the chamber is connected to the nozzle.

7. The method of claim 1, wherein a pressure for propelling the composition through the nozzle ranges from about 100 pounds per square inch (PSI) to about 1000 PSI.

8. The method of claim 7, wherein the pressure is about 200 PSI.

9. The method of claim 7, wherein the pressure is about 500 PSI.

10. The method of claim 1, wherein the compressed gas further comprises nitrogen or an inert gas.

11. The method of claim 10, wherein the inert gas is helium.

12. The method of claim 1, wherein the compressed gas further comprises air.

13. The method of claim 1, wherein the agent is selected from the group consisting of a nucleic acid, a protein, a lipid, a small molecule, a bacterium, and a virus.

14. The method of claim 1, wherein the MOF is a Cu-based MOF, a Fe-based MOF, a Ni-based MOF, a Co-based MOF, a Zn-based MOF, or a Mg-based MOF.

15. The method of claim 1, wherein the MOF is a zeolitic imidazolate framework (ZIF).

16. The method of claim 15, wherein the MOF is ZIF-8.

17. The method of claim 1, wherein the agent is encapsulated within the MOF.

18. The method of claim 1, wherein the composition further comprises a pharmaceutically acceptable polymer.

19. The method of claim 18, wherein the polymer comprises one or more of polylactic acid (PLA), poly (lactic-co-glycolic acid) (PLGA), tannin, and a starch-based polymer.

20. The method of claim 1, wherein the cell is a plant cell.

21. The method of claim 1, wherein the cell is a mammalian cell.

22. The method of claim 21, wherein the mammalian cell is a skin cell.

23. A system for introducing an agent into a cell, comprising:
a composition comprising an agent and a metal organic framework (MOF);
a nozzle configured to retain the composition therein; and
compressed gas comprising CO₂ retained within a chamber that is connected to the nozzle.

24.-42. (canceled)

43. A method of introducing an agent into a cell, comprising introducing the agent into the cell using the system of claim 23.
